(19) United States
(12) Patent Application Publication
(10) Pub. No.: US 2015/0224305 A1
(43) Pub. Date: Aug. 13, 2015

(54) Method to enhance orthodontic tooth movement

(71) Applicant: Rapid Orthodontics Plus, Inc., San Diego, CA (US)

(72) Inventors: Zeev Davidovitch, Columbus, OH (US); Robert Sanford, Hampton, NJ (US); Moshe Davidovitch, Tel Aviv (IL); Jacob Dagan, Tel Aviv (IL); Shlomo Assa, San Diego, CA (US)

(73) Assignee: Rapid Orthodontics Plus, Inc., San Diego, CA (US)

(21) Appl. No.: 14/177,573

(22) Filed: Feb. 11, 2014

(51) Int. Cl. A61N 1/20; A61C 7/14; A61C 7/20; A61C 7/08

(52) U.S. Cl. CPC A61N 1/205; A61C 7/08; A61C 7/14; A61C 7/20

(57) ABSTRACT

A method and apparatus to improve and accelerate orthodontic tooth movement and alveolar bone apposition and resorption, constituted of a device providing biocompatible contact anode in contact with gingival epithelia at the area of osteoclastic activity, and biocompatible contact cathode in contact with gingival epithelia at the area of osteoblastic activity, arranged to apply electric current across the anode and the cathode to stimulate alveolar bone remodeling. The method and apparatus provide for an electronic circuit that is pre-programmed via wireless communication to adjust the electric current magnitude and time duration so as to enhance tooth movement, oseteogenesis, and osteoclasia.

This invention relates to orthodontic techniques in general, and the reduction in time required for specific tooth movements in particular.

### BACKGROUND OF THE INVENTION

For well over a century, orthodontists have been engaged in the process of repositioning teeth from a bad relationship, or “malocclusion,” into a healthier and more esthetic arrangement. In order to move teeth, three elements are generally required: 1) force, 2) time and 3) space. The mouth responds to a sustained force placed on a tooth by rearranging, or “remodeling,” the jawbone around the root of the tooth. This remodeling creates space around the tooth allowing the tooth to move in the direction of the force. Not only does the tooth need space within the jawbone, but it is also imperative to have or create spacing between the teeth in order for movement to occur.

Over the years, orthodontists have invented devices, generally referred to as an “appliance,” that permit clinicians to deliver sustained forces to the teeth. Braces, or “orthodontic brackets and arch wires,” are the classic appliances that most, if not all, orthodontists use. Braces consist of small brackets that are glued, or “bonded,” to the crowns of teeth, and a wire is then inserted into slots in the brackets and held into place with a ligature or clip. The brackets do not generate forces themselves, but rather transfer forces to the teeth from the deflected wire, when it is inserted into the slot on the bracket and held in place by the ligature. The wire has a “memory,” i.e., a characteristic by which the wire tends to return to its original shape, and in doing so, exerts a force on the bracket that is in turn transmitted to the tooth. Through the application of various types, shapes and sizes of wires, the teeth eventually align themselves into a more ideal occlusion. The technical term used among orthodontists to describe braces is “comprehensive fixed appliance.”

Because the orthodontic treatment takes a long time, and of the obvious drawbacks of having foreign objects glued to a patient's teeth, appliances have been heretofore developed that can be inserted and removed by the patient, and worn part-time. A myriad of removable appliances have been developed over the years, but the vast majority of them are not “comprehensive” in nature. In other words, the removable appliances address specific movements or malocclusions, and are only used for a certain limited period of time. Treatment with removable appliances is often used in conjunction with braces or other appliances.

As mentioned above, Orthodontic tooth movement presently is accomplished by the application of mechanical forces to teeth. An apparatus is connected inside the mouth of a patient which applies, through the use of springs, rubber bands, composite material casting apparatus or other means, a mechanical force in the direction of desired tooth movement. These forces cause the bone to resorb (be removed) in the direction of force and cause the bone to grow on the other side of the tooth, to fill-up the space created by the movement of the dental root in the direction of the force.

This process of orthodontic force application enables teeth to move in the mouth within the boundaries of the neighboring teeth and tissues. The tooth movement is clarified by Wolffs Law which states, in effect, that bone under mechanical stress is remodeled to accommodate and reduce the stress. The common practical aspect to known techniques of orthodontic movement is that the mechanical apparatus, commonly referred to as “braces,” must be worn by the patient for extended periods of time, often several years or more in order to achieve the desired results. The classic orthodontic tooth movement treatment that requires the continuous application of forces to create the necessary tooth movement is expensive as it requires frequent modifications of the magnitude and direction of forces applied to different teeth to achieve the necessary results, requiring frequent adjustments by the treating Orthodontist. Moreover, wearing the mechanical fixtures known as “braces” creates a considerable discomfort for the patient, and at the same time this condition will cause an aesthetic concern to the patient as the mechanical fixtures (Braces) are visible to other people. In addition, the braces enable the accumulation of bacteria and viruses, harmful to the teeth and their surrounding tissues. For these reasons, it is very desirable to shorten the duration of orthodontic treatment in each and every case.

U.S. Pat. No. 3,842,841 teaches the application of a direct current to aid healing of bone fractures in the human body, but requires surgical implantation. A negative electrode (cathode) is surgically inserted into the site of a fracture, and a positive electrode (anode) is taped to the skin elsewhere. The precise biological process is complex; and the current flowing through the fractured bone increases the healing rate of the damaged bone tissue thus reducing the healing time of the injury, enabling the patient to return to normal life in many instances much faster relative to the classical treatment that is not using direct current as used in the prior art above.

U.S. Pat. No. 4,153,060 teaches a method and apparatus for electrically stimulating bone growth and tooth movement in the mouths of humans. A positive electrode is placed on the gum surface adjacent the bone structure which is to be resorbed. A negative electrode is placed on the gum surface adjacent the bone tissue which is to be accreted or built up. A current source is connected, such that a small current flows between the electrodes, which have the effect of stimulating bone growth in a specific direction. In a particular arrangement, the electrodes are placed on the gum surface adjacent a tooth, the positive electrode on the side towards which the tooth should move, and the negative on the side from which the tooth will move. Application of a small current to the electrodes will enhance the repositioning of the tooth in conjunction with normal orthodontic practices.

U.S. Pat. No. 4,854,865 teaches an improved method of orthodontic electro-osteogenesis using a biocompatible anode in contact with an electrolytic gel between the anode and epithelial gingiva at an area of osteoclastic or osteoblastic activity, and a biocompatible cathode in contact with a different type of electrolytic gel between the cathode and epithelial gingiva at an area of osteoclastic or osteoblastic activity. Electric current is then applied across the anode and cathode to stimulate osteogenesis. This method stimulates osteogenesis, which is a crucial element in tooth movement but is unable to demonstrate how to achieve desirable results, or to enable to complete orthodontic treatment in a shorter amount of time.

To date, there have been no recognizable inventions that demonstrate improvements in enhancing tooth movement, non-invasively, with the ability to effect one tooth or many teeth, to reduce the total amount of time over which an orthodontic appliance must be used in order to accomplish a given amount of tooth movement or repositioning.

It is therefore an object of this invention to provide a method to accelerate tooth movement which will shorten the overall time a patient has to wear an orthodontic appliance system which will overcome the drawbacks of the prior art.

### SUMMARY OF THE INVENTION

Therefore, in view of the foregoing, it is an object of the present invention to reposition teeth in a patient's mouth by applying an electrical potential to the patient's gums in the immediate vicinity.

It is a further object of the present invention to increase the rate of movement of teeth undergoing mechanical stress in accordance with known orthodontic practices.

Actors in this operation are cells capable of responding to a circuit mounted in an housing that will be placed in conjunction with any type of existing orthodontic appliance for providing the necessary electric current output to electrodes located adjacent to a tooth to be positioned, in such a manner that electric current polarity, electric current direction and electric field strength will all increase the rate of teeth movement undergoing mechanical stress in accordance with known orthodontic practices.

It is an additional object of the present invention to provide a method and apparatus for stimulating and controlling bone growth in a patient's mouth in order to correct alveolar bone defects, close cleft palates, or maintain the alveolar ridge in edentulous patients (those who have lost their teeth).

In accordance with the above, the other objects, a method and apparatus for the initiation and enhancement of tooth movement comprises the disposition of an anodic electrode of a particular size and shape in the direction of applied force and a cathodic electronic of a particular size and shape on the opposite side of the tooth to be moved. A current source is connected to the two electrodes which causes the tooth to be repositioned in combination with an existing orthodontic appliance.

In accordance with the above, yet another objective of the present invention will be to benefit from the ability to administer simultaneously two or more methods to cells capable of responding to an exposure to each method alone from the biological, cellular and molecular stand point of tooth structure. The present invention will further show that the combined application of tooth pushing force and electric current together, which evokes additive or synergistic responses by the affected cells, allows the treating Orthodontist to reduce the dose of each of the stimulating factors in order to achieve an optimal response. This principle means that the addition of exogenous electric currents, which is the principle of the present invention, to the orthodontic mechanical force will enable the treating Orthodontist to reduce the amount of mechanical pressure necessary to achieve the desired results, and by following the teachings of the present invention will reduce the risk of root resorption associated with the application of orthodontic force alone.

The novelty and usefulness of this invention is that the application of a specific electric current, through appropriate surface electrodes in the mouth, also can be utilized to stimulate bone accretion in the vicinity of a cathodic electrode and bone resorption in the vicinity of an anodic electrode, and that this biological stimulation accelerates the velocity of orthodontic tooth movement.

In accordance with the above, yet another objective of the present invention will be the augmentation of orthodontic anchorage non-invasively applying an electrical potential to the patient's gums in the immediate vicinity. Modern orthodontics is a field of medicine based on an understanding of growth and development, and is implemented by biomechanics. The biological response to the mechanical force(s) applied to the teeth will determine changes in their position. Inherent in this mode of therapy is Newton's third law of physics which states that for every action there is an equal and opposite reaction. A common example in orthodontics illustrating this point is when protruding upper front teeth are retracted against the posterior teeth (referred to as the anchorage segment/unit) of either arch, which causes the latter to be protracted in reaction. This is not always a desirable side effect and attempts to negate it are numerous, and are based on altering the balance on the mechanics side of the equation. However, our ever increasing understanding of the biology of tooth movement has offered the clinician a non-invasive simplistic method of altering this imbalance on the biological side of the equation. To achieve this, the specific made and fit appliance to a patient's mouth comprising the application of a specific electric current, through appropriate surface electrodes in the mouth, can be directed (with specifically placed electrodes), to cause bone apposition in areas to where tooth movement needs to be minimized, while causing bone resorption where movement is desired simply by changing the direction (polarity) of the flow of current in each of these areas. This effect can be modified according to the stage of treatment for the individual patient by programming the above mentioned specific appliance to perform the above mentioned function.

### BRIEF DESCRIPTION OF THE DRAWINGS

The invention will now be described with reference to the accompanying drawing which is a plain illustrative view of the apparatus which operates in accordance with the method for the invention.

FIG. 1 is a schematic illustration of a longitudinal section of a human mandibular canine.

FIG. 2 is a schematic illustration of some physical changes that result from application of an orthodontic force to a tooth.

FIG. 3 is a schematic presentation of the rationale for the location of the electrodes on the gingival tissues near teeth undergoing orthodontic treatment.

FIG. 4 illustrates the electric field strength between Anode and Cathode made at the same size.

FIG. 5 illustrates the electric field strength between Anode and Cathode made at different size.

FIG. 6 illustrates a simplified orthodontics particular tooth movement required by the repositioning teeth into a healthier and more esthetic arrangement.

FIG. 7 illustrates a simplified orthodontic particular tooth movement required by the repositioning teeth and the required placement of anode and cathode for accelerating tooth movement.

FIG. 8 illustrates a simplified orthodontic particular tooth rotational movement required by the repositioning teeth and the required placement of anodes and cathodes for accelerating tooth movement.

FIG. 9 illustrates orthodontic personalized brace that places the Anodes and Cathodes based on the particular tooth movement required by the repositioning of teeth into a healthier and more esthetic arrangement.

FIG. 10 illustrates orthodontics personalized brace that places the Anodes and Cathodes based on the particular tooth movement mounted On the patient jaw.

### DETAILED DESCRIPTION OF THE INVENTION

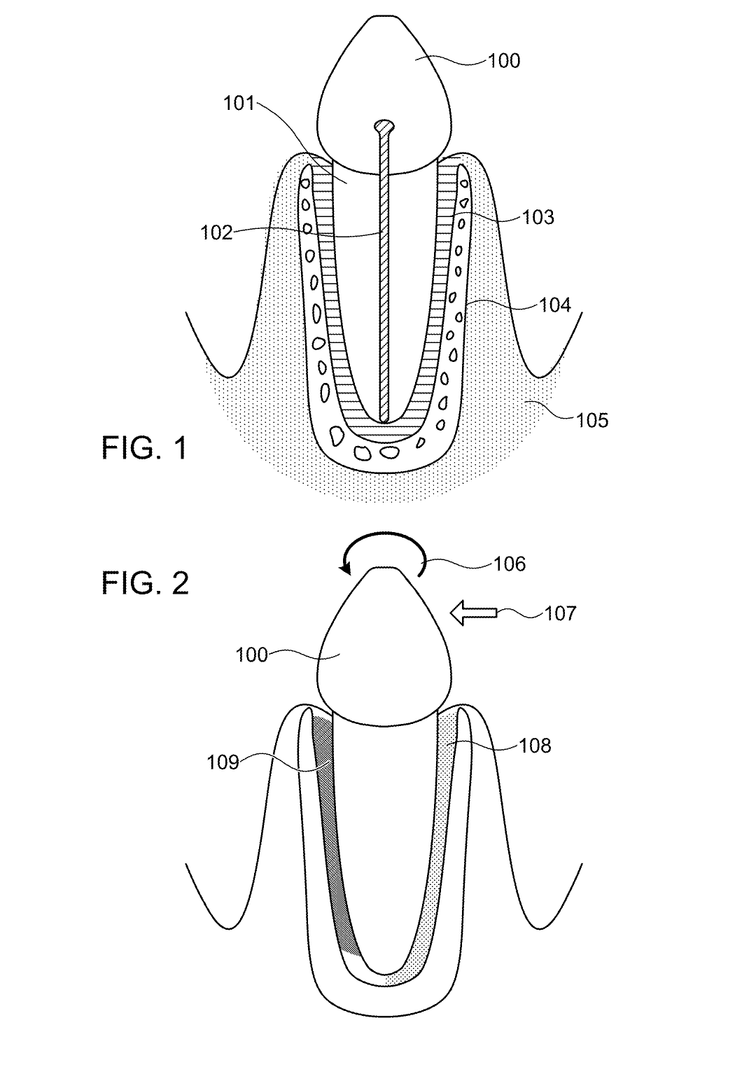

Referring to FIG. 1., there is shown a schematic illustration of a longitudinal section of a human mandibular canine. The tooth is comprised of a crown 100 and a root 101. Centrally located within the body of the tooth is its root canal 102. The crown is visible in the mouth, while the root is not, because it is encased in soft connective tissue fibrous mesh known as the periodontal ligament (PDL) 103, and the PDL is surrounded by alveolar bone 104. The alveolar bone faces the PDL on one side, and is covered by the gingival and mucosal tissue (gum) 105 on the other. All these tissues, dental and paradental, contain living cells, which are responsible for the remodeling of all these tissues in response to orthodontic forces. This remodeling process is the mechanism that facilitates orthodontic tooth movement.

Referring to FIG. 2, there is shown a schematic illustration of some physical changes that result from application of an orthodontic force to a tooth 100. The mechanical forces and moments are applied to affect the desired type of tooth movement. Accordingly, a tooth may be extruded, intruded, rotated, tipped, or translated. This figure illustrates a translatory movement which results from a translational force 107 and a rotation 106 of the crown around a center of rotation, or fulcrum, located near the apex of the root. In order to insure a translatory movement a force couple is created opposite to the rotational force 106. This force couple (generated by the interaction between the bracket and the arch wire for braces, and attachments and aligners for Invisalign) creates a moment that moves the root along with the crown extending the center of rotation from the apex to infinity. The translational force 107 literally pushes the root against the alveolar wall of bone 104, opposite to the point of force application, compressing the PDL 109. Likewise, on the side of the tooth where the force 107 is being applied, the PDL is being stretched 108. In the latter case, the dental root 101 is displaced within the alveolar bone dental socket 104, and the PDL 103 responds by widening 108 and compressing 109. In the latter case, the dental root 101 is displaced within the alveolar bone dental socket 104, and the PDL 103 is stretched on the aspect of the dental root being distanced from the alveolar bone 108. In contrast, the opposing aspect 101, pressure is created in the PDL 109 as a result of approximating the root 101 toward the alveolar bone 104.

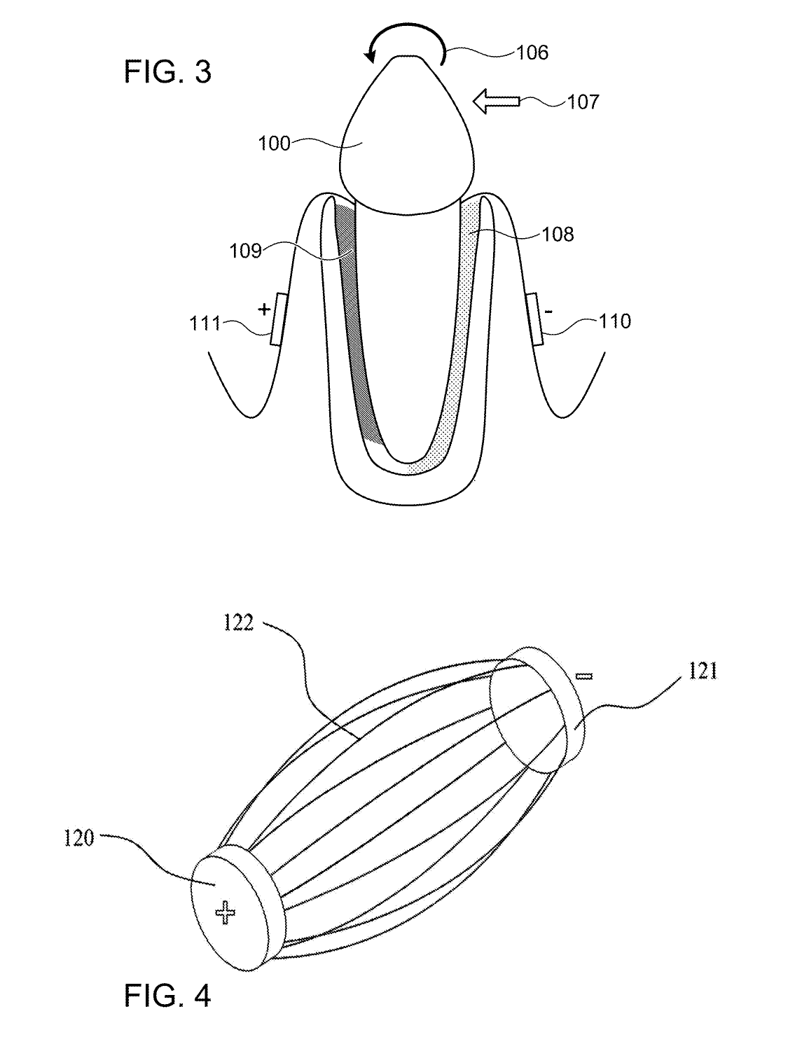

FIG. 3, there is a schematic presentation of the rationale for the location of the electrodes on the gingival tissues near teeth undergoing orthodontic treatment. When a tooth is undergoing translatory movement, as depicted in FIG. 2, the alveolar bone facing the compressed PDL 109 undergoes removal (resorption), while in zones where the PDL is stretched 108, new layers of bone are deposited on the surface of the old alveolar bone. Over time, the alveolar bone socket 104 remodels, allowing the tooth to assume a new position in the place that the translational force 107 caused it to move. This remodeling is a direct result of, and consistent with, the compression/resorption and stretching/deposition activity in the PDL and juxtaposed alveolar bone. When alveolar bone is pressed upon, as occurs when a translational force 107 presses on a tooth and the PDL compresses, a short-lived electrical spike can be measured across the alveolar 104 bone's matrix. This is known as a piezoelectric effect, characterized by the negative side (cathode) of the potential being detected on the concave side of the flexed bone and that contain positive side (anode) of the potential being detected on the convex side. In addition, the mechanical stress causes movement of tissue fluids. These fluids contain electrical charges that change the cellular electric polarity, stimulating the cells to remodel their surrounding matrices. These stress-generated streaming potentials (SGP) last about 20-30 minutes. When the bone is held in a flexed state, the concave side experiences bone deposition and the convex side experiences bone resorption. As long as the bone remains flexed, this process continues over time until the previously flexed bone appears unflexed or straight. Our research has revealed that by applying minute electrical direct currents to oral tissues, the underlying bone exhibits similar patterns of deposition (cathode 110) and resporption (anode 111) observed following the application of orthodontic treatment. In this manner, simultaneous applications of mechanical forces and electric current will augment the apposition and resorption of bone, thereby reducing the resistance of the bone, enhancing translation and accelerating the velocity of tooth movement.

FIG. 4. shows a schematic presentation of an anode 120 placed in parallel to a cathode 121 at a particular distance, and when electric voltage is applied on the said anode 120 and cathode 121, the electric current that flows through the media between the anode 120 and cathode 121 will form an electric field that the equi-potential lines 122 will be as shown in FIG. 4 herein. The distance between anode 120 and cathode 121 and the media in between will influence the curvature of the electric field 122. In this preferred embodiment the use of identical size anode and cathode will limit the volume of human tissue that is influenced by the electric field and electric current that is in between the anode and cathode.

FIG. 5 shows a schematic presentation of a large diameter size anode 123 placed in parallel to a smaller diameter size cathode 124 at a particular distance, and when electric voltage is applied on the said larger anode 123 and smaller size cathode 124, the electric current flow through the media in between the anode 123 and cathode 124 will form an electric field that the equi-potential lines 125 will be as shown in FIG. 5 herein. The distance between the larger size anode 123 and smaller cathode 124 and the media in between will influence the curvature of the electric field 125. In this yet another preferred embodiment, the use of a larger anode 124 with a smaller size cathode 125 will increase the volume of human tissue that is influenced by the electric field and electric current that is in between the anode and cathode. In yet another preferred embodiment the anode 123 will be of a smaller diameter size while the cathode 124 will be of a larger diameter size to change the electric field influence on the human tissue that is placed between the anode 123 and cathode 124.

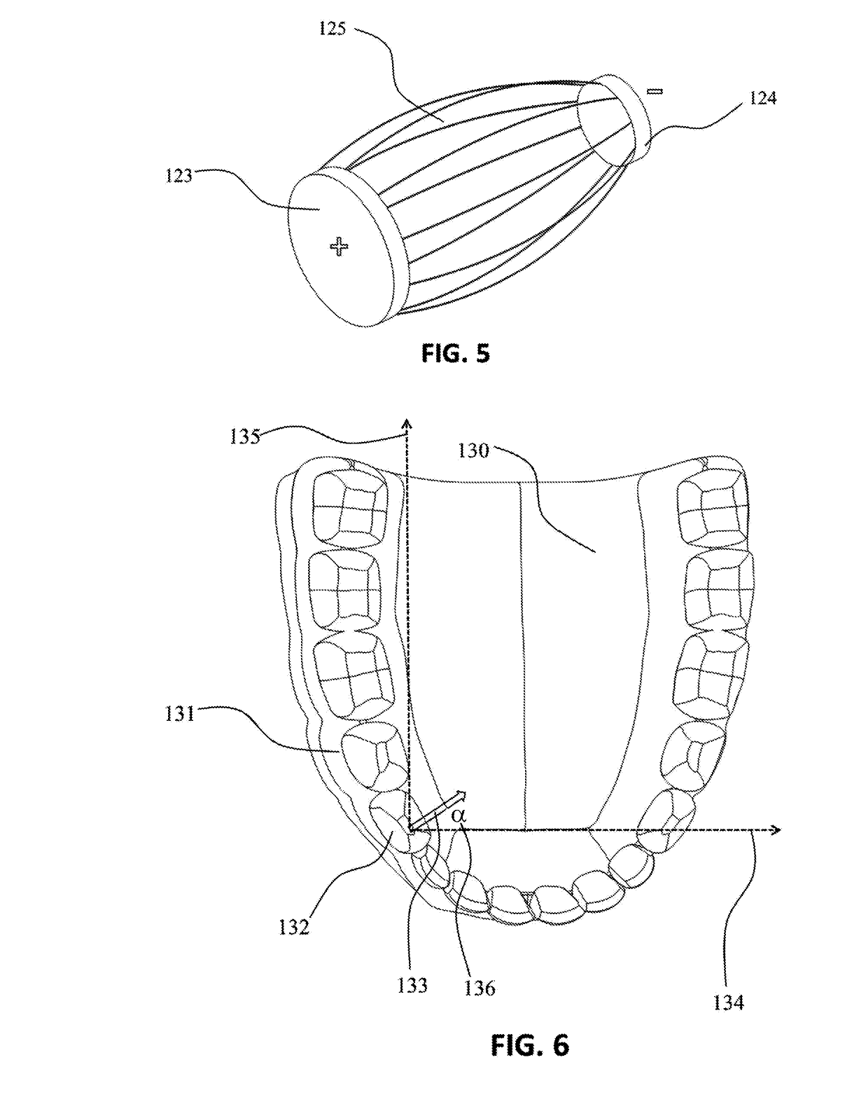

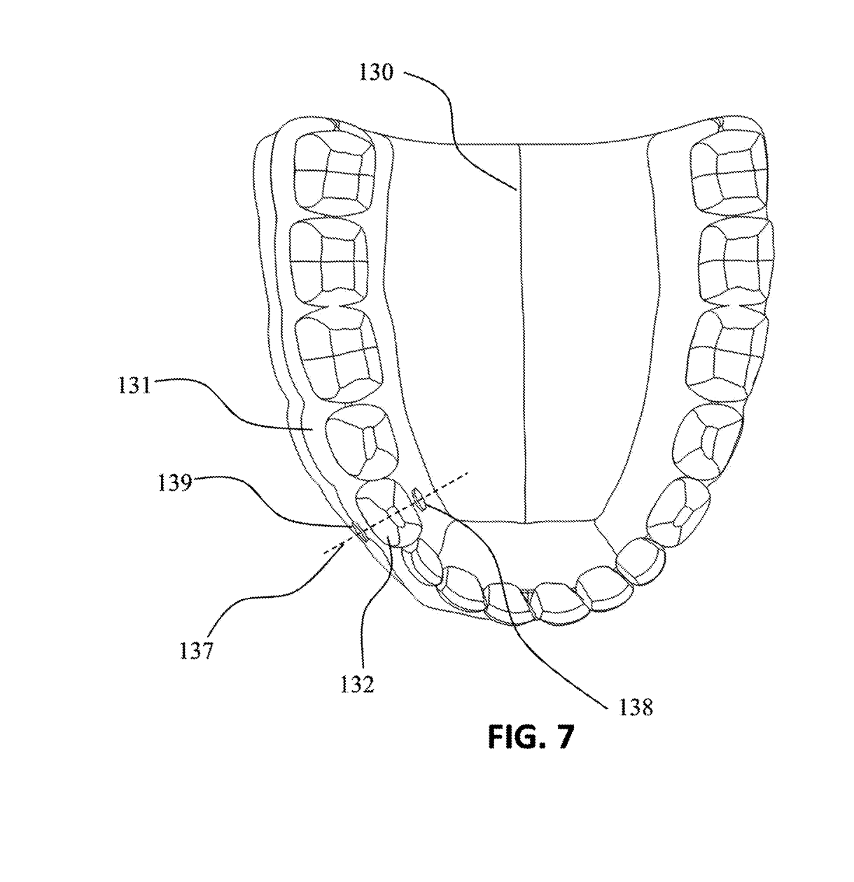

FIG. 6 is a schematic presentation of a human lower jaw 130, with the gum tissue 131, where the canine tooth 132 needs to be moved in the direction 133 shown herein. The need to move the canine tooth 132 is determined by an Orthodontist that is treating a patient in the process of repositioning teeth from a bad relationship, or “malocclusion,” into a healthier and more esthetic arrangement. In this preferred embodiment the use of canine tooth is for illustration and for explanation of this invention. Referring to FIG. 6., 135 is the vertical axis that is parallel to the center line of jaw, 131, while 134 is the orthogonal axis that will be referred to as the x axis. The desired canine tooth movement vector 133 can be defined by an angle 136 in relation to a jaw x axis 134. In this preferred embodiment the Orthodontist will define the corrective need to move the canine tooth at a magnitude defined by vector 133, measured in millimeters, and the movement direction in relations to x axis 136 with angle 133 measured in degrees.

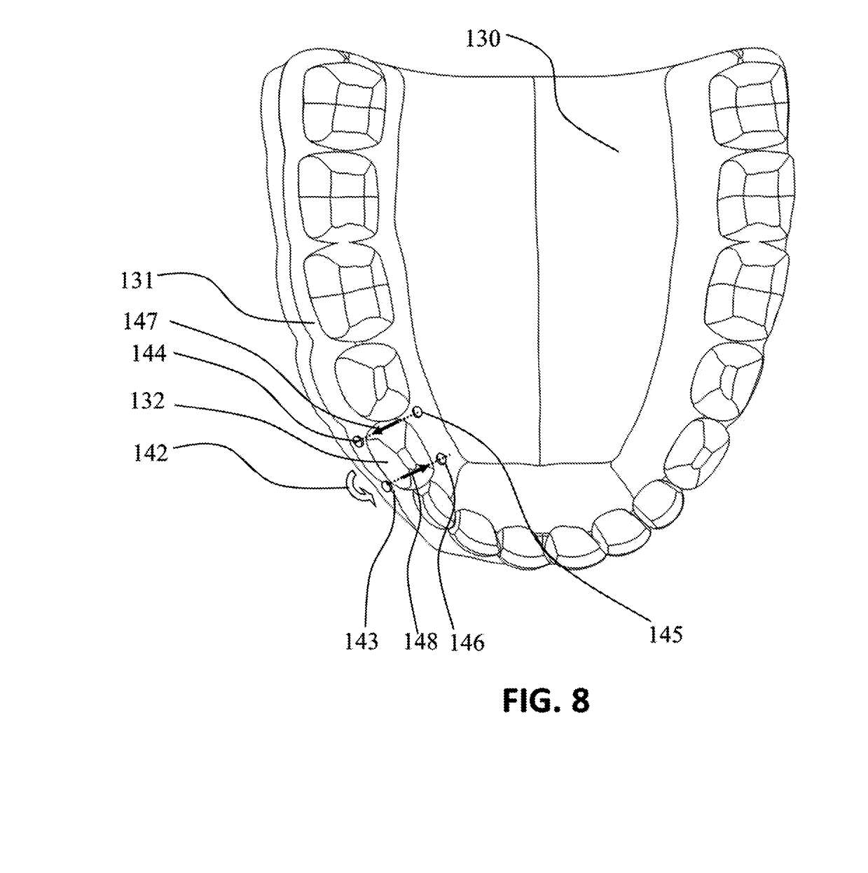

FIG. 7 is a schematic presentation of a human lower jaw 130, with the gum tissue 131, where the canine tooth 132 needs to be moved at the direction 133 shown in FIG. 6 above. The need to move the canine tooth 132 is determined by an Orthodontist who is treating a patient in the process of repositioning teeth from a bad relationship, or “malocclusion,” into a healthier and more esthetic arrangement. In this preferred embodiment the use of the canine tooth is for illustration and for explanation of this invention. Referring to FIG. 7, the direction of moving canine tooth 132 shown by vector 133 of FIG. 6 above will create the imaginary center line 137 shown in FIG. 8 herein. In this preferred embodiment, the imaginary center line that is aligned and parallel to the canine tooth desired movement shown in 133 of FIG. 6, will be used to place cathode 139 at the labial (lip) side of the gum line 131, whereas the anode 138 will be placed at the lingual (tongue) side of the gum tissue 131. The centerline of the cathode 139 and anode 138 will be parallel and aligned with the desired canine tooth movement 133 of FIG. 6 above. In this preferred embodiment the anode 138 and cathode 139 are of the same diameter and size to guide the electric current activity at the root of the canine tooth. In yet another preferred embodiment the cathode 139 will be of a larger diameter size whereas the anode 138 will be at a smaller diameter size to distribute the electric field over larger area in the canine tooth root. And yet in another preferred embodiment, the anode 138 will be of a larger diameter size, whereas the cathode 139 will be of a smaller diameter size to distribute the electric field over larger area in the canine tooth root. In this preferred embodiment the polarity of the electric filed which is set by the placement of the anode 138 and the cathode 139 will be perfectly aligned and parallel with the axis 137 that is parallel and aligned with the canine tooth desired movement vector 133 of FIG. 6 above. In yet another preferred embodiment where the canine desired tooth movement vector 133 of FIG. 6 above will be parallel to 133 of FIG. 6 above but in an opposite direction, the placement of anode 138 and cathode 139 will be different, such that in this yet another preferred embodiment where the canine desired tooth movement vector 133 of FIG. 6 is 180 degrees opposite in direction from what is shown in FIG. 6 above, than in this yet another preferred embodiment the anode will be placed at 139 shown in FIG. 7 and the cathode will be placed at 138 to support the opposite desired canine tooth movement.

In this preferred embodiment the electric current characteristics will be of a Direct Current with the capabilities to regulate and deliver constant current of 20 micro-Amperes (20 millionth of a one Ampere current) for duration of 4 hours, while the treated patient will wear the appliance 140 of FIG. 8 below. In yet another preferred embodiment, the Direct Current will be varied constantly in a range of 15-25 micro-Amperes for durations that will be 3 to 5 hours. In this preferred embodiment, the appliance 140 of FIG. 8 below, will be recharged during the time that it will not be in use, preferably during day time, so the appliance 140 of FIG. 8 would be used by the treated patient during the night hours while sleeping. In this preferred embodiment, the electronic circuit will be preprogrammed by the treating Orthodontist to operate for 4 hours and to regulate both the Direct Current voltage and the electric current to be 20 micro-Amperes.

FIG. 8 is a schematic presentation of a human lower jaw 130, with the gum tissue 131, where the canine tooth 132 needs to be moved at the rotational direction 142 shown herein, the need to move the canine tooth 132 is determined by an Orthodontist who is treating a patient in the process of repositioning teeth from a bad relationship, or “malocclusion,” into a healthier and more esthetic arrangement. In this preferred embodiment the use of the canine tooth is for illustration and for explanation of this invention. Referring to FIG. 8, in this another preferred embodiment, the direction of rotational movement of canine tooth 132 shown by motion symbol 142, will create the imaginary center line 147 shown in FIG. 8 herein, representing canine tooth 132 movement of one side as part of the rotational canine tooth movement, and the other imaginary center line 148 shown in FIG. 8 herein, representing the canine tooth 132 movement on the other side as part of the rotational movement of the canine tooth 132 shown herein. In this preferred embodiment, the 2 imaginary center lines that are aligned and parallel to the canine tooth 132 desired rotational movement, will be used to place cathodes 143 and anode 144 at the labial (lip) side of the gum line 131, whereas the anodes 146 and cathode 145 will be placed at the lingual (tongue) side of the gum tissue 131. The centerline of the cathode 143 and anode 146 will be parallel and aligned with the desired canine tooth rotational movement 142 of FIG. 8 herein, and at the same time the center line of cathode 145 and anode 144 will be parallel and aligned with the desired canine tooth rotational movement 142 of FIG. 8 herein.

In this preferred embodiment the anodes 144 and 146 and cathode 143 and 145 are of the same diameter and size to guide the electric current activity at the root of the canine tooth. In yet another preferred embodiment the cathodes 144 and 146 will be of a larger diameter size whereas the anodes 143 and 145 will be at a smaller diameter size to distribute the electric field over larger area in the canine tooth root. And yet in another preferred embodiment, the anodes 143 and 145 will be of a larger diameter size, whereas the cathodes 144 and 146 will be of a smaller diameter size to distribute the electric field over larger area in the canine tooth root. In this preferred embodiment the polarity of the electric filed which is set by the placement of the anodes 143 and 145 and the cathodes 144 and 146 will be perfectly aligned and parallel with the axis of rotation defined by symbol 142 that are parallel and aligned with the canine tooth desired rotational movement direction 142 shown herein. In yet another preferred embodiment where the canine desired tooth rotational movement will be in an opposite direction to rotational direction 142 shown herein, the placement of anodes 143 and 145 and cathodes 144 and 146 will be different, such that the anodes will be placed at 144 and 146 shown in FIG. 8 and the cathodes will be placed at 143 and 145 to support the opposite desired canine rotational tooth movement.

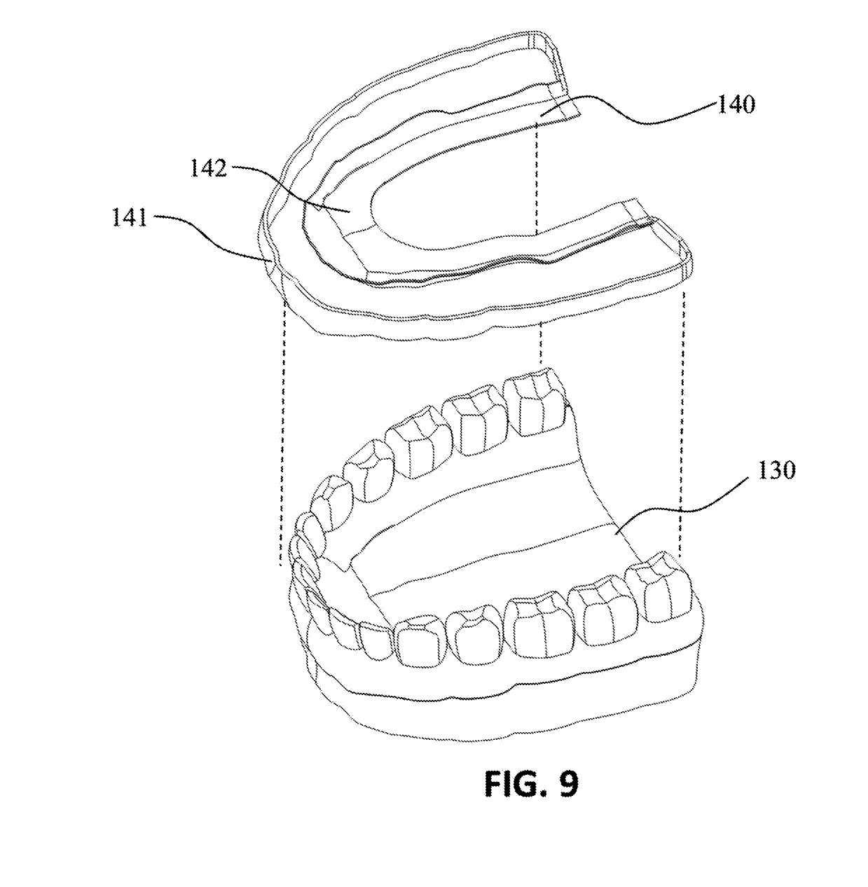

In this preferred embodiment the electric current characteristics will be of a Direct Current with the capabilities to regulate and deliver constant current of 20 micro-Amperes (20 millionth of a one Ampere current) for duration of 4 hours, while the treated patient will wear the appliance 140 of FIG. 9 below. In yet another preferred embodiment, the Direct Current will be varied constantly in a range of 15-25 micro-Amperes for durations that will be 3 to 5 hours. In this preferred embodiment, the appliance 140 of FIG. 9 below will be recharged during the time that it will not be in use, preferably during day time, so the appliance 140 of FIG. 9 would be used by the treated patient during the night hours while sleeping. In this preferred embodiment, the electronic circuit will be preprogrammed by the treating Orthodontist to operate for 4 hours and to regulate both the Direct Current voltage and the electric current to be 20 micro-Amperes.

FIG. 9. is a schematic presentation of a complete appliance 140, prepared to fit a particular patient's lower jaw 130 herein. In this preferred embodiment in the appliance 140 provided a section 142 which holds the anode 138 of FIG. 7 above, connected to an internal electronic circuit that provides the positive voltage connected to the anode 138 of FIG. 6 above. Also, in this preferred embodiment in the appliance 140 provided a section 141 which holds the cathode 139 of FIG. 7 above, connected to an internal electronic circuit that provides the positive voltage connected to the cathode 139 of FIG. 6 above. In this preferred embodiment the appliance 140 will provide the location and the means to contact the anode 138 of FIG. 7 above and the correct location, and the appliance 140 will provide the location and the means to contact the cathode 139 of FIG. 7 above and the correct location so the centerline of the anode 138 and cathode 139 137 of FIG. 7 will be perfectly aligned and parallel with the canine desired tooth movement vector 133 of FIG. 6 above.

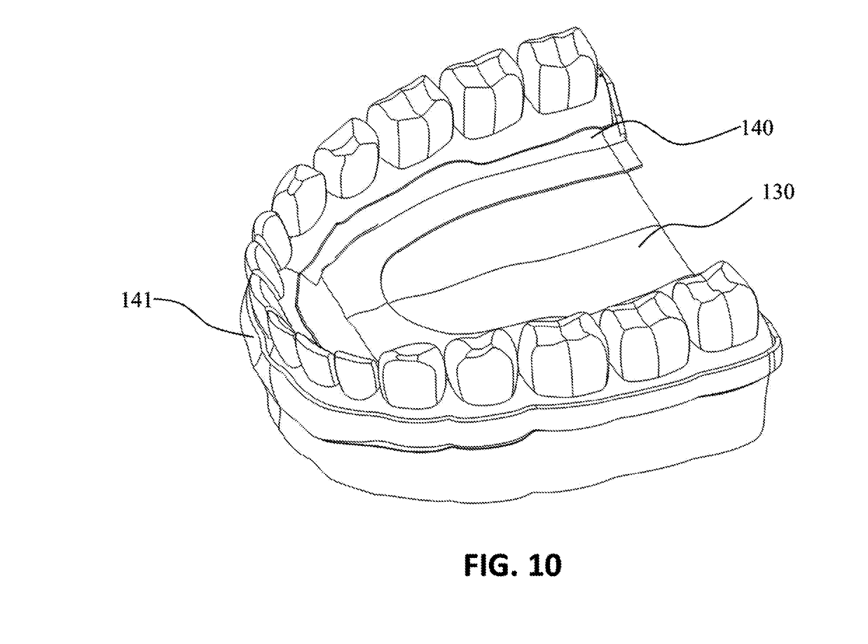

FIG. 10 is a schematic presentation of a complete appliance 140 that is mounted on the patient's jaw 130.

1-20. (canceled)

21. An appliance arranged to fit a jaw of a patient, said appliance comprising:
an orthodontic appliance arranged to provide a rotational orthodontic force to a tooth having a root in the jaw of the patient, said rotational orthodontic force arranged to generate rotational motion of the tooth, said generated rotational motion defining:
a first motion vector in a first direction at a portion of a first side of the tooth, said first direction from a lingual side of a gum tissue of the jaw of the patient to a labial side of the gum tissue of the jaw of the patient; and
a second motion vector, in a second direction opposing said first direction, at a portion of a second side of the tooth, said second side of the tooth opposing said first side of the tooth;
an electronic circuit;
a first cathode associated with the tooth, said first cathode responsive to said electronic circuit and arranged to contact the lingual side of the gum tissue of the jaw of the patient juxtaposed with the first side of the tooth;
a first anode associated with the tooth, said first anode responsive to said electronic circuit and arranged to contact the labial side of the gum tissue of the jaw of the patient juxtaposed with the first side of the tooth of the tooth to be rotated, a first centerline extending between said first anode and said first cathode parallel and aligned with the first motion vector;
a second cathode associated with the tooth, said second cathode responsive to said electronic circuit and arranged to contact the labial side of the gum tissue of the jaw of the patient juxtaposed with the second side of the tooth to be rotated; and
a second anode associated with the tooth, said second anode responsive to said electronic circuit and arranged to contact the lingual side of the gum tissue of the jaw of the patient juxtaposed with the second side of the tooth, a second centerline extending between said second anode and said second cathode parallel and aligned with the second motion vector,
wherein said electronic circuit is configured to generate:
a first direct current flow between said first anode and said first cathode; and
a second direct current flow between said second anode and said second cathode, wherein the polarity of said first direct current flow and said second direct current flow are arranged to assist in bone resorption and deposition in accordance with the provided rotational orthodontic force.

22. The appliance according to claim 21, wherein each of said first direct current flow and said second direct current flow are in the range of 15-25 microamperes.

23. The appliance according to claim 22, wherein the amount of said first and second direct current flows are programmable.

24. The appliance according to claim 22, wherein said electronic circuit is further arranged to be programmed so as to:
provide said first and second direct current flows for a predetermined duration of between 3 and 5 hours; and
not provide said first and second direct current flows at the expiration of said predetermined duration.

25. The appliance according to claim 21, wherein said electronic circuit is further arranged to be programmed so as to:
provide said first and second direct current flows for a predetermined duration of between 3 and 5 hours; and
not provide said first and second direct current flows at the expiration of said predetermined duration.

26. A method of rotating a tooth in a jaw of a patient, the method comprising:
providing a rotational orthodontic force to the tooth via an orthodontic appliance, said provided rotational orthodontic force defining:
a first motion vector in a first direction at a portion of a first side of the tooth, said first direction from a lingual side of a gum tissue of the jaw of the patient to a labial side of the gum tissue of the jaw of the patient; and
a second motion vector, in a second direction opposing said first direction, at a portion of a second side of the tooth, said second side of the tooth opposing said first side of the tooth;
providing a first cathode associated with the tooth, said provided first cathode juxtaposed with the first side of the tooth and in contact with the lingual side of the gum tissue of the jaw of the patient;
providing a first anode associated with the tooth, said provided first anode juxtaposed with the first side of the tooth and in contact with the labial side of the gum tissue of the jaw of the patient, and further arranged such that a first centerline extends between said first anode and said first cathode parallel and aligned with the first motion vector;
providing a second cathode associated with the tooth, said provided second cathode juxtaposed with the second side of the tooth and in contact with the labial side of the gum tissue of the jaw of the patient;
providing a second anode associated with the tooth, said provided second anode juxtaposed with the second side of the tooth and in contact with the lingual side of the gum tissue of the jaw of the patient, and further arranged such that a second centerline extends between said second anode and said second cathode parallel and aligned with the second motion vector;
generating a first direct current flow between said provided first anode and said provided first cathode and a second direct current flow between said provided second anode and said provided second cathode so as to assist in bone resorption and deposition in accordance with the provided rotational orthodontic force.

27. The method according to claim 26, wherein each of said generated first direct current flow and said second direct current flow are in the range of 15-25 microamperes.

28. The method according to claim 27, wherein the amount of said first and second direct current flows are programmable.

29. The method according to claim 27, further comprising:
providing said first and second direct current flows for a predetermined duration of between 3 and 5 hours; and
not providing said first and second currents at the expiration of said predetermined duration.

30. The method according to claim 26, further comprising:
providing said first and second direct current flows for a predetermined duration of between 3 and 5 hours; and
not providing said first and second currents at the expiration of said predetermined duration.
